(19) United States
(12) Plant Patent Application Publication
(10) Pub. No.: US 2021/0068323 P1
(43) Pub. Date: Mar. 4, 2021

(54) Climbing rosa plant named 'Poulcy040'

(50) ;

(71) Applicant: Mogens Nyegaard Olesen, Fredensborg (DK)

(72) Inventor: Mogens Nyegaard Olesen, Fredensborg (DK)

(21) Appl. No.: 16/602,254

(22) Filed: Sep. 4, 2019

(51) Int. Cl. A01H 6/74

(52) U.S. Cl. PLT/115

(57) ABSTRACT

A new garden rose plant of the climbing class which has abundant, red flowers and attractive foliage. This new and distinct variety has shown to be uniform and stable in the resulting generations from asexual propagation.

Botanical designation: Rosa hybrid.

Variety denomination: ‘Poulcy040’.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2008 and the resulting seeds were planted in a controlled environment in Fredensborg, Denmark. The new variety, named ‘Poulcy040’, originated as a single seedling from the stated cross.

The new variety may be distinguished from its male pollen parent and female seed parent primarily by the following characteristics. The male pollen parent plant has a bushy growth habit of a compact floribunda, while the new variety has a climbing and spreading growth habit. The female seed parent plant has deep pink flowers while the new variety has red flowers.

The objective of the hybridization of this rose variety was to create a new and distinct variety with unique qualities, such as:

1. Uniform and abundant red flowers;

2. Vigorous, but compact growth when propagated on its own roots;

3. Exceptional disease resistance; and

4. Reduced apical dominance in flowering habit. The new variety consistently produces flowers evenly from the lower branches to the top of the plant.

This combination of qualities is not present in previously available commercial cultivars of this type, known to the inventor, and distinguish ‘Poulcy040’ from all other varieties of which we are aware.

As part of the rose development program, Mogens N. Olesen germinated the seeds from the aforementioned hybridization during winter of 2008 and conducted evaluations on the resulting seedlings in a controlled environment in Fredensborg, Denmark. ‘Poulcy040’ was selected in the spring of 2009 by the inventor as a single plant from the progeny of the aforementioned hybridization.

Asexual reproduction of ‘Poulcy040’ by rooted cuttings was first done by Mogens N. Olesen in the nursery in Fredensborg, Denmark in July, 2009. This initial and other subsequent asexual propagations conducted in controlled environments have demonstrated that the characteristics of ‘Poulcy040’ are true to type and are transmitted from one generation to the next.

### DESCRIPTION OF THE DRAWING

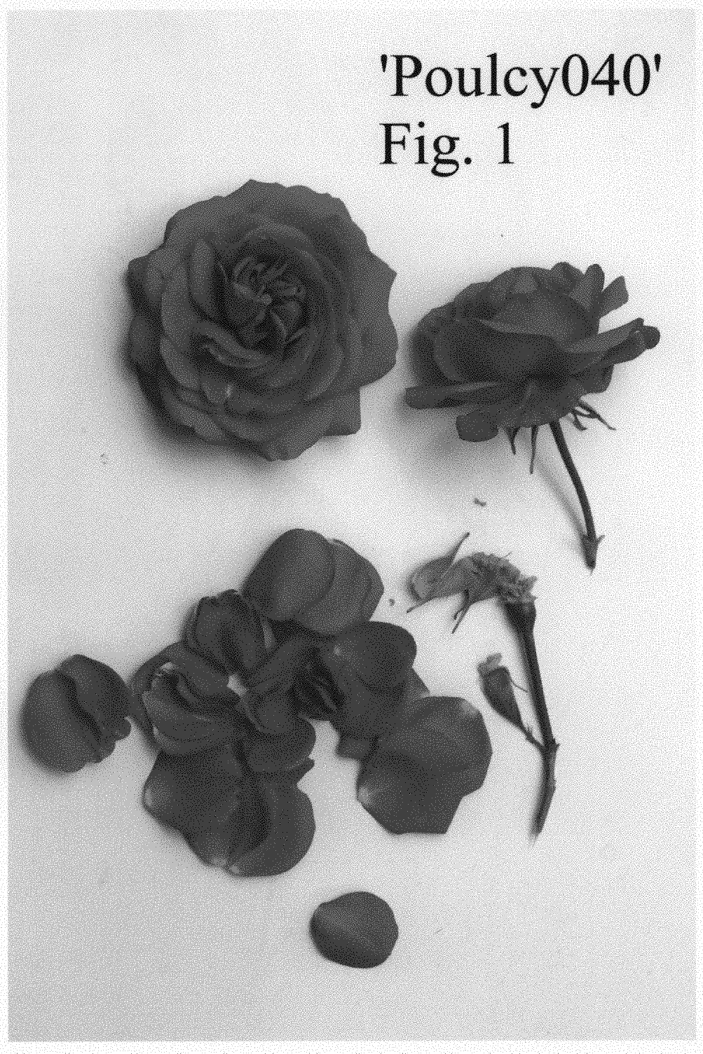

The accompanying color illustrations show as true as is reasonably possible to obtain in color photographs of this type, the typical characteristics of the buds, flowers, leaves, and stems, of ‘Poulcy040’. Specifically illustrated in FIG. 1 of the drawings are fully opened flowers, viewed from above and the side, detached flower petals, detached sepals, showing pistils, anthers, and filaments.

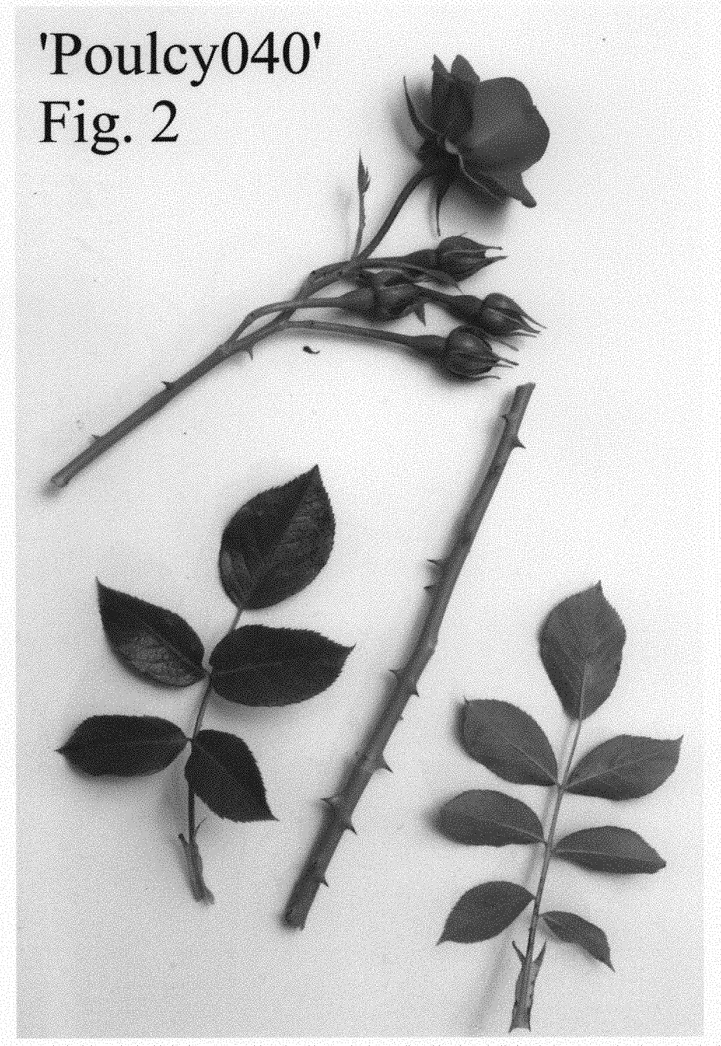

Specifically illustrated in FIG. 2 of the drawings are stems, leaves, and a cluster of flowers on a branch. Plants shown are 2 years of age.

### DETAILED DESCRIPTION OF THE VARIETY

The following is a description of ‘Poulcy040’, as observed in its growth in a field nursery in Linn County, Oreg. Observed plants are 2 years of age, and were grown on their own roots. Color references are made using The Royal Horticultural Society (London, England) Colour Chart, 2001, except where common terms of color are used.

For a comparison, several physical characteristics of the rose variety ‘Poulyc009’, U.S. Plant Pat. No. 15,411 are compared to ‘Poulcy040’ in Chart 1.

CHART 1

| | ‘Poulcy040’ | ‘Poulyc009’ |
|---|---|---|
| Petal Count | 75 petals | 25 petals |
| Flower Diameter | 75 mm | 70 mm |
| General Tonality | Red Group | Red-Purple |
| of Flower Color | 52A | 58B to 58C |

### FLOWER AND FLOWER BUD

Blooming habit: Continuous.
Flower bud:
   Size.—Upon opening, 23 mm in length from base of receptacle to end of bud. Bud diameter is generally 14 mm.
   Bud form.—Globose.
   Bud color.—As sepals divide petals are distinctly Red Group 45A and Greyed-Purple Group 187A.
   Sepal inner surface.—Color: Yellow-Green Group 146D. Surface: Lightly pubescent.
   Sepal outer surface.—Color: Yellow-Green Group 144A with intonations of Greyed-Purple Group 183A. Texture: Smooth.
   Sepal shape.—Apex: Cirrhose. Base: Flat at union with receptacle.
   Sepal margin.—Margins have weak foliaceous appendages on three of the five sepals.
   Sepal size.—30 mm long, 10 mm wide.
   Receptacle.—Texture: Smooth. Size: 10 mm in height, 7 mm wide. Color: Yellow-Green Group 144A with intonations of Greyed-Purple Group 183B. Shape: Globular.
   Pedicel.—Surface: Smooth. Length: 35 mm. Diameter: 3 mm on average. Color: Yellow-Green Group 144A with intonations of Greyed-Orange Group 175A. Strength: Strong.
   Peduncle.—Length: 15 to 35 cm. Diameter: About 5 mm. Color: Yellow-Green Group 144A. Texture: Smooth. Observations: Prickles Greyed-Yellow Group 162A.
Flower bud development: Flower buds are borne in clusters of 5 flower buds per stem on average.
Flower bloom:
   Fragrance.—Moderate.
   Duration.—The blooms have a duration on the plant of approximately 14 days. Petals fall cleanly away from plant after flowers have fully matured.
   Size.—Flower diameter is 75 mm when open. Flower depth is 35 mm.
   Flower shape.—Very double rosette with overlapping petals and a tightly closed center.
   Shape of flower, side view.—The upper portion is flat convex.

The lower portion is flat concave.
Petalage: Under normal conditions, flowers have about 75 petals.
General tonality of flower: Open flowers are Red Group 52A.
Petal color:
   Upon opening, outer petals.—Upper surface: Red Group 52A. Lower surface: Red-Purple Group N57A.
   Upon opening, inner petals.—Upper surface: Red Group 52A. Lower surface: Red-Purple Group N57A.
   Basal petal spots, upon opening.—Upper surface: Yellow Group 4D. Lower surface: Yellow Group 4D.
   After opening, outer petals.—Upper surface: Red Group 52A. Lower surface: Red-Purple Group N57A.
   After opening, inner petals.—Upper surface: Red Group 52A. Lower surface: Red-Purple Group N57A.
   Basal petal spots, after opening.—Upper surface: Yellow Group 4D. Lower surface: Yellow Group 4D.
Petals:
   Petal reflex.—Somewhat reflexed.
   Margin.—Entire and uniform. Moderate or very light undulations.
   Shape.—Broad and elliptic. Apex shape: Rounded. Base shape: Broadly acute.
   Size.—35 mm (1)×38 mm (w).
   Texture.—Smooth.
   Thickness.—Average.
Petaloids:
   Size.—20 mm (1) by 15 mm (w).
   Quantity.—About 15.
   Shape.—Elliptical with an acute base and acute apices.
   Color.—Upper surface is Red Group 45B, underneath Red Group 50A. Yellow 4D at the petaloid base.
Reproductive flower parts:
   Pollen.—None observed.
   Anthers.—Size: 1.5 mm in length. Color: Yellow Group 8A. Quantity: 44 on average.
   Filaments.—Color: Yellow Group 8C. Length: 10 mm.
   Pistils.—Length: 6 mm. Quantity: 21 on average.
   Stigmas.—Color: Yellow-White 158A.
   Styles.—Color: Yellow-White 158A.
   Location of stigmas.—Level in location relative to the length of the filaments and the height of the anthers.
   Hips.—None Observed.
Plant growth: Climbing and spreading. Plants are 90 to 150 cm in height, and 75 cm wide.
Stems:
   Color of juvenile growth.—Yellow-Green Group 144B.
   Color of mature growth.—Yellow-Green Group 144A.
   Length.—Canes are about 70 cm from the base of the plant to the flowering portion.
   Diameter.—About 5 mm.
   Internodes.—On mature canes about 35 mm between nodes.
   Surface texture.—Young wood: Smooth. Older wood: Smooth.
Long prickles:
   Incidence.—10 prickles per 10 cm of stem.
   Size.—Average length of prickles on mature stems is 9 mm.
   Shape.—Upper portion is linear. Lower portion is concave.
   Color.—Juvenile prickles: Greyed Yellow Group 160A. Mature prickles: Greyed Yellow Group 160A.
Plant foliage:
   Compound leaf.—160 mm (1)×100 (w).
   Quantity.—2 leaves per 10 cm of stem on average.
   Leaf bearing angle to the stem.—45 degrees.
   Color of juvenile foliage.—Upper side: Yellow-Green Group 146A. Lower side: Yellow-Green Group 146B.
   Color of mature foliage.—Upper side: Yellow-Green Group 147A. Lower side: Yellow-Green Group 146B.
Plant leaves and leaflets:
   Stipules.—Size: 31 mm long, 5 mm wide. Quantity: 2 per compound leaf. Shape: Linear, slightly broad based with outward extending apices. Margins: Finely serrated. Color: Yellow-Green Group 144B.
   Petiole.—Length: 31 mm. Diameter: 2 mm. Upper surface color: Yellow-Green Group 144A. Lower surface color: Yellow-Green Group 144A.
   Rachis.—Length: 80 mm. Upper surface color: Yellow-Green Group 144A. Lower surface color: Yellow-Green Group 144A.
   Leaflet.—Quantity: 5 to 7 leaflets. Margins: Serrated. Size: Terminal leaflets are about 52 mm long, 35 mm wide. Shape: Generally elliptical. Base: Rounded, partially oblique. Apex: Mucronate. Texture: Smooth. Thickness: Average. Arrangement: Odd pinnate. Venation: Reticulate. Glossiness: Very glossy.
Disease resistance: Above average resistance to powdery mildew Sphaerotheca pannosa, downy mildew Peronospora sparsa, rust Phragmidium sps., black spot Diplocarpon rosae, and Botrytis cinerea under normal growing conditions.
Cold hardiness: The variety is tolerant to USDA Cold Hardiness Zone 6.
Heat tolerance: The variety has been found to be suitable for climate conditions found in the American Horticulture Society heat zone 7.

We claim:

1. A new and distinct variety of rose plant of the climbing rose class named ‘Poulcy040’, substantially as illustrated and described herein, due to its abundant red flowers, disease resistance, and extended period of bloom.
